(19) United States
(12) Patent Application Publication
(10) Pub. No.: US 2026/0069724 A1
(43) Pub. Date: Mar. 12, 2026

(54) METHODS AND MATERIALS FOR MAKING PET RADIOTRACERS

(71) Applicant: CellSight Technologies Inc., San Francisco, CA (US)

(72) Inventors: Jelena Levi, San Francisco, CA (US); Ananth Srinivasan, El Dorado Hills, CA (US); Alexander Hoepping, Radeberg (DE); Rene Smits, Radeberg (DE)

(73) Assignee: CellSight Technologies Inc., San Francisco, CA (US)

(21) Appl. No.: 19/383,258

(22) Filed: Nov. 7, 2025

Related U.S. Application Data
(63) Continuation of application No. 17/314,366, filed on May 7, 2021, now abandoned.

(51) Int. Cl. A61K 51/04; C07B 59/00; C07H 19/16

(52) U.S. Cl. CPC A61K 51/0491; C07B 59/005; C07H 19/16; C07B 2200/05

(57) ABSTRACT

Embodiments of the present disclosure provide compositions comprising compounds useful to make radiotracers for positron emission tomography imaging, as well as methods for making and using these compositions.

### CROSS-REFERENCE TO RELATED APPLICATIONS

This application is a continuation application which claims the benefit under 35 U.S.C. § 120 and § 121 of U.S. patent application Ser. No. 17/314,366, filed May 7, 2021, the contents of which are incorporated herein by reference.

### BACKGROUND

The instant disclosure relates, in general, to compositions and methods for performing positron emission tomography (PET) and, more particularly, to compositions and methods for the development and use of ¹⁸F-based PET tracers useful in PET imaging techniques. The long physical half-life (109 minutes) of ¹⁸F-based tracers is very desirable as it allows for clinical studies without the necessity of an on-site cyclotron. In addition, with contemporary PET camera technologies, quantitative measurements of radioactivity concentration can be made with high temporal sampling and good statistical precision.

There is a need in the art for additional compositions and methods that are designed to make ¹⁸F-based PET tracers useful in PET imaging techniques. The development of new efficient methods and materials for making ¹⁸F-labeled tracers useful in positron emission tomography methods will provide artisans with greater opportunities to examine a range of clinical phenomena.

### SUMMARY

The invention disclosed herein has a number of embodiments. Embodiments of the invention include, for example, compositions of matter comprising a compound having the general formula:

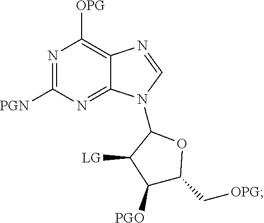

wherein: PG comprises a protecting group; and LG comprises a leaving group. Typically in such embodiments of the invention, the nitrogen atom coupled to the protecting group is coupled to two protecting groups as would be represented by “N(PG)₂”. Alternatively, this nitrogen atom is coupled to a hydrogen atom and a single protecting group as would be represented by “NHPG”. In illustrative embodiments of the invention, the composition includes at least one of precursor 1 or precursor 3:

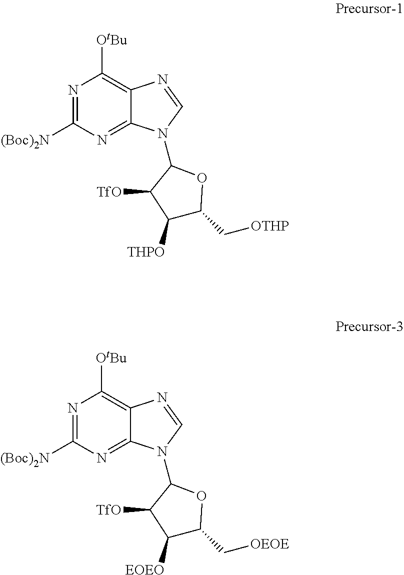

wherein: ^{t}Bu comprises a tert-Butyloxycarbonyl alcohol protecting group; Boc comprises a tert-Butyloxycarbonyl amine protecting group; THP comprises a tetrahydropyranyl alcohol protecting group; EOE comprises an ethoxyethyl alcohol protecting group; and Tf comprises a triflate leaving group.

The compositions of the invention can comprise a number of formulations. For example, in some embodiments of the invention, the compound having the general formula shown above (e.g., precursor 1 and/or precursor 3) is not disposed in a solvent. In other embodiments of the invention, the compound is disposed in a solvent, for example one comprising at least one of: acetonitrile, dimethyl sulfoxide, dimethylformamide, tertamyl alcohol, tetrahydrofuran, dioxane, and sulfone. In certain embodiments of the invention, the composition consists essentially of the compound. In some embodiments of the invention, the composition consists essentially of the compound disposed in a solvent.

In certain embodiments of the invention, the composition further comprises an isotope selected from the group consisting of: ¹⁸F, ¹³¹I, ¹²⁵I, ¹²⁴I, ¹²³I, ¹²¹I, ⁷⁷Br, and ⁷⁵Br. In some embodiments of the invention, the composition comprises a solvent such as acetonitrile, and an additional compound such as at least one of K₂CO₃, Na₂CO₃, potassium oxalate, or tetraethylammonium bicarbonate. In certain embodiments of the invention, the composition comprises an acid such as at least one of: HCl, HF, HBr, HI, or acetic acid. In certain embodiments of the invention, the composition comprises ¹⁸F AraG:

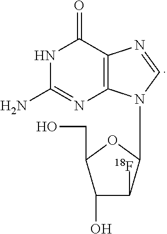

Other embodiments of the invention include methods of making a PET probe composition, the method comprising reacting a compound including an isotope (Ist) with a compound having the general formula:

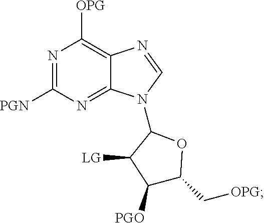

wherein said reacting removes the PG and LG moieties and couples the isotope to the compound; so that the PET probe composition is made. Typically, the compound is precursor 1 or precursor 3 and the PET probe made in this reaction comprises ¹⁸F AraG:

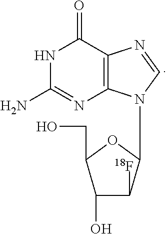

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIGS. 1A-1C show schematics of illustrative methods for preparing precursor compounds of the invention. FIG. 1A provides a schematic showing an illustrative procedure for the preparation of compound 6, which is a common starting point/compound for making both precursor 1 and precursor 3. FIG. 1B provides a schematic showing an illustrative procedure for making precursor 1 from compound 6. FIG. 1C provides a schematic showing an illustrative procedure for making precursor 3 from compound 6.

FIG. 2 shows a schematic of precursor 1 and precursor 3 used in in a radiosynthesis general scheme to make a PET radiotracer according to art accepted practices (see, e.g., Kang et al., J Label Compd Radiopharm 2006; 49: 1237-1246).

FIG. 3 shows graphed data from the results of radiosynthesis studies on the precursors of the invention. The top panel in FIG. 3 shows data from chromatograms of ¹⁸F AraG: using an UV-detector (254 nm); and the bottom panel shows ¹⁸F AraG: radioactivity trace. In these radiosynthesis studies, ¹⁸F- was eluted with 2 mg K2CO3/13 mg K222, azeotropic drying. Followed by the addition of 2 mg precursor in 0.5 mL MeCN, and then reaction at 100° C. for 10 min; the addition of 1 mL 1M HCl, reaction at 85° C. for 10 min, and then neutralization with 1 mL 1M NaOH yield ¹⁸F AraG.

### DETAILED DESCRIPTION

Before the present disclosure is described in greater detail, it is to be understood that this disclosure is not limited to particular embodiments described, and the embodiment of the invention as such may, of course, vary. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs. All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of synthetic organic chemistry, chemistry, and the like, which are within the skill of the art. Such techniques are explained fully in the literature. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used herein, the singular forms “a,” “an,” and “the” include plural referents unless the context clearly dictates otherwise. Thus, for example, reference to “a compound” includes a plurality of compounds. In this specification and in the claims that follow, reference will be made to a number of terms that shall be defined to have the following meanings unless a contrary intention is apparent.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. Further, documents or references cited in this text (e.g., U.S. patent Publication Nos. 20150230762, 20150297760 and 20190054198, 20210030878, 20200405667, 20190255002, 20160355460, 20160176807, 20150232415, 20150152206, 20140309424, 20120053337, 20100022746, 20100016551, 20090095635, 20090036668, U.S. U.S. Pat. No. 9,011,817; Jud and Micura, Chemistry 2017 23(14) 3406-3413; and Kang et al., J Label Compd Radiopharm 2006; 49: 1237-1246) and each of these documents or references (“herein cited references”), as well as each document or reference cited in each of the herein-cited references (including any manufacturer's specifications, instructions, etc.) are hereby expressly incorporated herein by reference.

As used herein, “alkyl” or “alkyl group” refers to a saturated aliphatic hydrocarbon radical which can be straight or branched, having 1 to 20 carbon atoms, wherein the stated range of carbon atoms includes each intervening integer individually, as well as sub-ranges. Examples of alkyl include, but are not limited to methyl, ethyl, n-propyl, i-propyl, n-butyl, s-butyl, t-butyl, n-pentyl, and s-pentyl. The term “lower alkyl” means an alkyl group having less than 10 carbon atoms.

The term “substituted,” as in “substituted alkyl”, “substituted phenyl,”
and the like means that the substituted group may contain in place of one or more hydrogens a group such as hydroxy, amino, halo, trifluoromethyl, cyano, —NH(lower alkyl), —N(lower alkyl)₂, lower alkoxy, lower alkylthio, or carboxy, and thus embraces the terms haloalkyl, alkoxy, fluorobenzyl, and the sulfur and phosphorous containing substitutions referred to below.

As used herein, “halo”, “halogen”, or “halogen radical” refers to a fluorine, chlorine, bromine, and iodine, and radicals thereof. Further, when used in compound words, such as “haloalkyl” or “haloalkenyl”, “halo” refers to an alkyl or alkenyl radical in which one or more hydrogens are substituted by halogen radicals. Examples of haloalkyl include, but are not limited to, trifluoromethyl, trichloromethyl, pentafluoroethyl, and pentachloroethyl.

The term “alkoxy” represents an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. The term “lower alkoxy” means an alkoxy group having less than 10 carbon atoms.

In accordance with the present disclosure, “a detectably effective amount” of embodiments of the present disclosure is defined as an amount sufficient to yield an acceptable image using equipment that is available for clinical use. A detectably effective amount of the embodiments of the present disclosure may be given in one or more administrations. The detectably effective amount in embodiments of the present disclosure can vary according to factors such as the degree of susceptibility of the individual, the age, sex, and weight of the individual, idiosyncratic responses of the individual, the dosimetry, and the like. Detectably effective amounts of embodiments of the present disclosure can also vary according to instrument and film-related factors. Optimization of such factors may bel within the level of skill in the art.

The term “detectable” refers to the ability to detect a signal or presence of an embodiment of the present disclosure over a background signal.

The term “detectable signal” or the phrases “detection of a labeled compound” or “detectable labeled compound” refers to the detection (directly or indirectly) of a labeled compound in a host or sample. The detection of a labeled compound refers to the ability to detect and distinguish the presence of a labeled compound in a host or sample from other background signals derived from the host or sample. In other words, there is a measurable and statistically significant difference (e.g., a statistically significant difference is enough of a difference to distinguish among the detectable signal and the background, such as about 0.1%, 1%, 3%, 5%, 10%, 15%, 20%, 25%, 30%, or 40% or more difference between the detectable signal and the background) between detectable signal and the background. Standards and/or calibration curves can be used to determine the relative intensity of the detectable signal and/or the background. The detectable signal can be generated from a small to large concentration of a labeled compound. In an embodiment, the detectable signal may need to be the sum of each of the individual labeled compound signals. In an embodiment, the detectable signal can be generated from a summation, an integration, or other mathematical process, formula, or algorithm. In an embodiment, the summation, the integration, or other mathematical process, formula, or algorithm can be used to process the detectable signal so that the detectable signal can be distinguished from background noise and the like.

As used herein, “agent”, “active agent”, or the like, can include a compound (e.g., labeled compound) of the present disclosure. The agent can be disposed in a composition or a pharmaceutical composition. As used herein, “pharmaceutical composition” refers to the combination of an active agent with a pharmaceutically acceptable carrier. As used herein, a “pharmaceutical composition” refers to a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, intravenous, buccal, rectal, parenteral, intraperitoneal, intradermal, intracheal, intramuscular, subcutaneous, inhalational and the like.

A “pharmaceutically acceptable excipient,” “pharmaceutically acceptable diluent,” “pharmaceutically acceptable carrier,” or “pharmaceutically acceptable adjuvant” means an excipient, diluent, carrier, and/or adjuvant that are useful in preparing a pharmaceutical composition that are generally safe, non-toxic and neither biologically nor otherwise undesirable, and include an excipient, diluent, carrier, and adjuvant that are acceptable for veterinary use and/or human pharmaceutical use. For compositions suitable for administration to humans, the term “excipient” is meant to include, but is not limited to, those ingredients described in Remington: The Science and Practice of Pharmacy, Lippincott Williams & Wilkins, 21st ed. (2006) the contents of which are incorporated by reference herein.

The term “unit dosage form,” as used herein, refers to physically discrete units suitable as unitary dosages for human and/or animal subjects, each unit containing a predetermined quantity of a compound calculated in an amount sufficient (e.g., weight of host, disease, severity of the disease, etc.) to produce the desired effect in association with a pharmaceutically acceptable diluent, carrier or vehicle. The specifications for unit dosage forms depend on the particular compound employed, the route and frequency of administration, and the effect to be achieved, and the pharmacodynamics associated with each compound in the host.

The term “effective amount” as used herein refers to that amount of an embodiment of the present disclosure (which may be referred to as a labeled compound) being administered that can be used to image a cell such as a heart cell.

By “administration” is meant introducing an embodiment of the present disclosure into a subject. Administration can include routes, such as, but not limited to, intravenous, oral, topical, subcutaneous, intraperitoneal, intraarterial, inhalation, vaginal, rectal, nasal, introduction into the cerebrospinal fluid, or instillation into body compartments can be used.

As used herein, the term “host” or “subject” includes humans, mammals (e.g., cats, dogs, horses, etc.), and other living animals. In particular, the host is a human subject. Typical hosts to which embodiments of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. Additionally, for in vitro applications, such as in vitro diagnostic and research applications, body fluids and cell samples of the above subjects will be suitable for use as a “sample”, such as mammalian (particularly primate such as human) blood, urine, or tissue samples.

The invention disclosed herein has a number of embodiments. Embodiments of the invention include, for example, compositions of matter comprising a compound having the general formula:

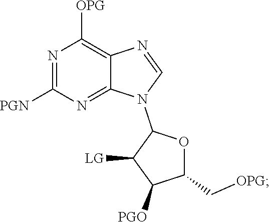

wherein: PG comprises a protecting group; and LG comprises a leaving group. Typically in this embodiment of the invention, the nitrogen atom coupled to the protecting group (e.g. a tert-Butyloxycarbonyl amine protecting group) is coupled to two protecting groups as would be represented by “N(PG)₂”. Alternatively in this embodiment of the invention, this nitrogen atom is coupled to a hydrogen atom and a single protecting group as would be represented by “NHPG”. In the schematic above, “PGN” encompasses both of these ways in which this nitrogen atom can be protected by one or more protecting groups. In illustrative embodiments of the invention, the composition includes at least one of precursor 1 or precursor 3:

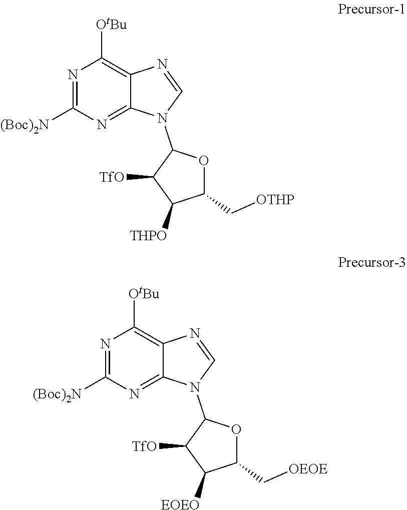

wherein: ^{t}Bu comprises a tert-Butyloxycarbonyl alcohol protecting group; Boc comprises a tert-Butyloxycarbonyl amine protecting group; THP comprises a tetrahydropyranyl alcohol protecting group; EOE comprises an ethoxyethyl alcohol protecting group; and Tf comprises a triflate leaving group.

The compositions of the invention can have a number of forms. For example, in some embodiments of the invention, the compound having the general formula (e.g., precursor 1 and precursor 3):

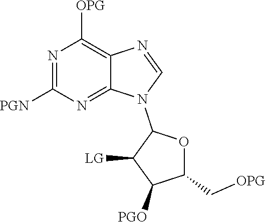

is not disposed in a solvent. In other embodiments of the invention, the compound is disposed in a solvent, for example one comprising at least one of: acetonitrile, dimethyl sulfoxide, dimethylformamide, tertamyl alcohol, tetrahydrofuran, dioxane, and sulfone. In certain embodiments of the invention, the composition consists essentially of the compound. In some embodiments of the invention, the composition consists essentially of the compound disposed in a solvent.

In certain embodiments of the invention, the composition further comprises an isotope selected from the group consisting of: ¹⁸F, ¹³¹I, ¹²⁵I, ¹²⁴I, ¹²³I, ¹²¹I, ⁷⁷Br, and ⁷⁵Br. In some embodiments of the invention, the composition comprises a solvent such as acetonitrile, and an additional compound such as at least one of K₂CO₃, Na₂CO₃, potassium oxalate, or tetraethylammonium bicarbonate. In certain embodiments of the invention, the composition comprises an acid such as at least one of: HCl, HF, HBr, HI, or acetic acid. In certain embodiments of the invention, the composition comprises ¹⁸F AraG:

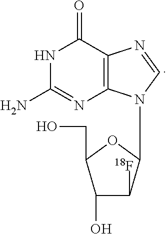

AraG is a nucleoside analog that has proven efficacy in the treatment of T cell lymphoblastic diseases. It is metabolized in a unique fashion by deoxyguanosine kinase. The methods and materials disclosed here can be used to synthesize the ¹⁸F AraG derivative to use as a molecular probe. Such probes can be used, for example, to examine the uptake and metabolism in cell lines and determine the efficacy of this compound in imaging a variety of human diseases.

In certain embodiments of the invention, amounts of the compound in the composition are at least 0.5 mg or at least 1.0 mg, for example from 2 mg to 15 mg. In certain embodiments of the invention, the composition is disposed in a kit comprising a vessel for storing the composition (e.g., one having a capacity less than 50 milliliters, 10 milliliters or 1 milliliter); and optionally a stabilizing agent (e.g., an inert gas such as N₂).

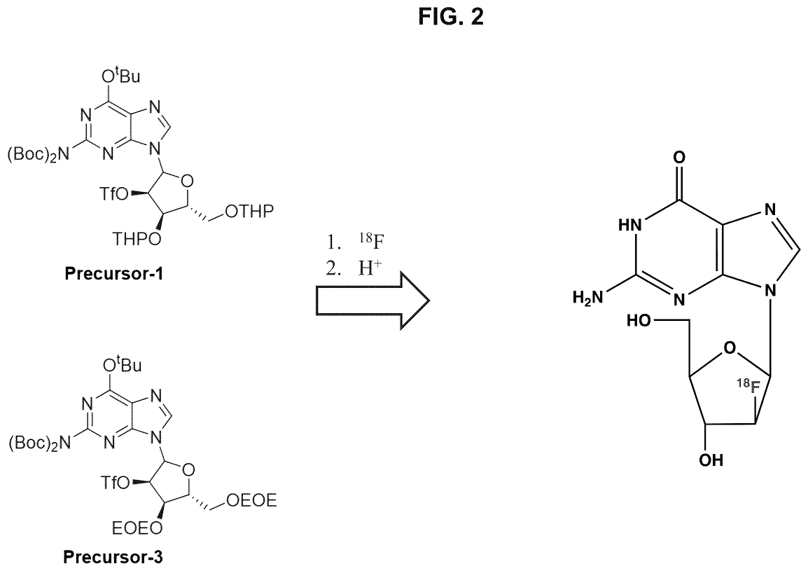

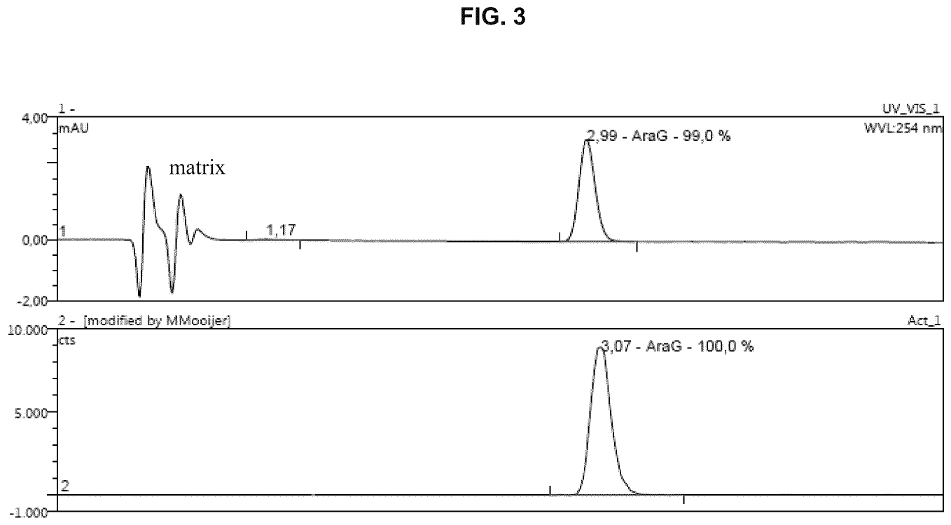

Other embodiments of the invention include methods of making a PET probe composition, the method comprising reacting a compound including an isotope (Ist) with a compound having the general formula (e.g., precursor 1 and precursor 3):

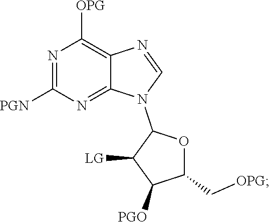

wherein said reacting removes the PG and LG moieties and couples the isotope to the compound; so that the PET probe composition is made. See, e.g., FIG. 2 and U.S. Pat. No. 9,011,817, the contents of which are incorporated herein by reference. Typically, this PET probe composition comprises:

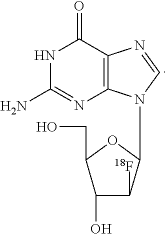

In certain methodological embodiments of the invention, the isotope is selected from the group consisting of: ¹⁸F, ¹³¹I, ¹²⁵I, ¹²⁴I, ¹²³I, ¹²¹I, ⁷⁷Br, and ⁷⁵Br. Optionally, for example, the isotope is selected from the group consisting of: [¹⁸F]KF, [¹³¹I]NaI, [¹²⁵I]NaI, [¹²⁴I]NaI, [¹²³I]NaI, [¹²¹I]NaI, [⁷⁷Br]NaBr, [⁷⁷Br]Br₂, [⁷⁵Br]NaBr, and [⁷⁵Br]Br₂. In certain embodiments of the invention, reacting occurs in a solvent selected from the group consisting of: dimethyl sulfoxide (DMSO), acetonitrile, dimethylformamide, and a combination thereof.

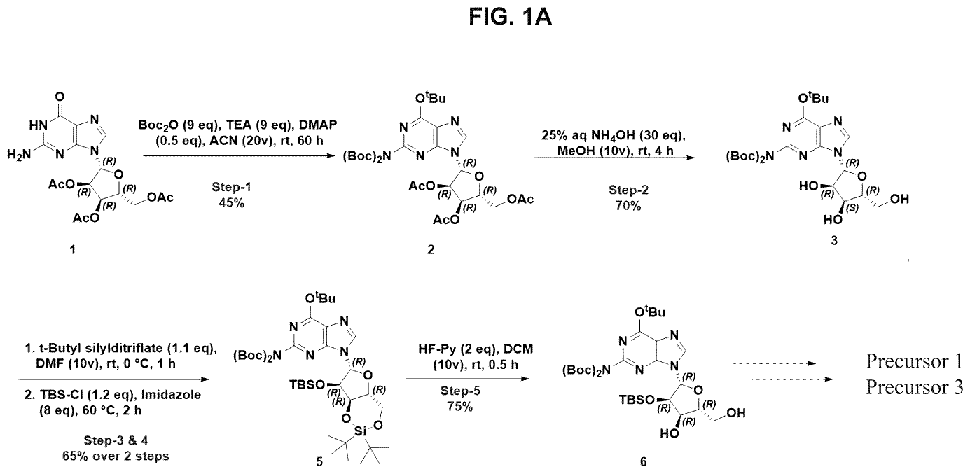

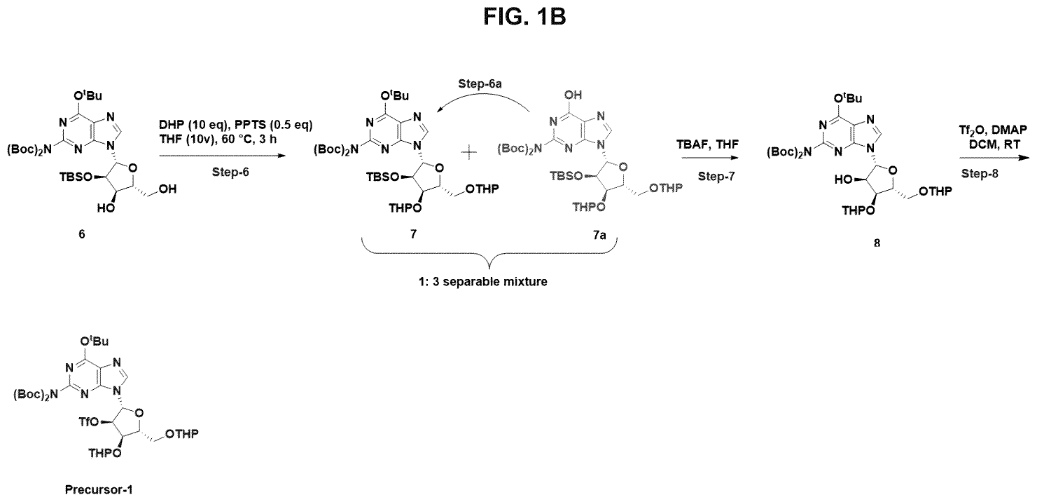

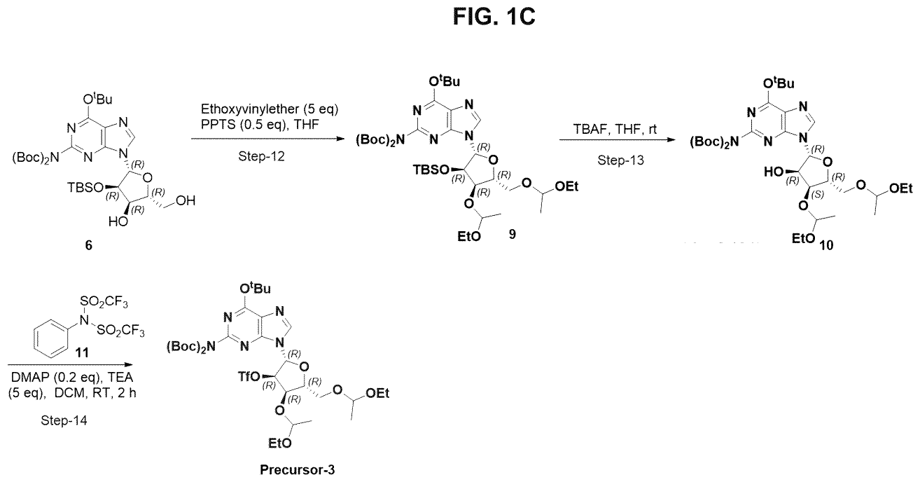

Another embodiment of the invention is a method of making compound 6, an illustrative example of which is shown in FIG. 1A. For example, embodiments of the invention include methods comprising using compound 1 as a starting material, and: Step 1. O6-tert-butyl, N2 (bis-[tert-butyloxycarbonyl]) protection of the guanine nucleobase; Step 2. Basic deprotection of acetyl protecting groups; Step 3. Protection of 3′ and 5′ OH groups with the di-tert-butylsilyl clamp followed by tert-butyldimethylsilyl protection of 2′ OH group (Step 4); and Step 5. Selective cleavage of 3′,5′ O-ditert-butylsilyl group gives compound 6, which is a common starting point for both precursors 1 and 3. See, e.g., Jud and Micura, Chemistry 2017 23(14) 3406-3413. Related embodiments include compositions of matter comprising compound 6, for example this compound disposed in a solvent.

Yet another embodiment of the invention is a method of making precursor 1 or precursor 3 (see, e.g., FIGS. 1A, 1B and 1C):

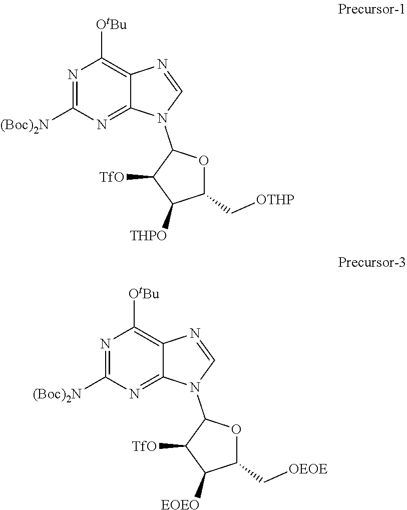

wherein: ^{t}Bu comprises a tert-Butyloxycarbonyl protecting group; Boc comprises a tert-Butyloxycarbonyl protecting group; THP comprises a tetrahydropyranyl protecting group; EOE comprises an ethoxyethyl protecting group; and Tf comprises a triflate leaving group, the method comprising using compound 6 as a starting material, and: protecting 3′ and 5′ OH groups of compound 6 using a tetrahydropyranyl ether; selectively deprotecting a tert-butyldimethylsilyl group of a compound formed in this step; and introducing a triflate leaving group in the 2′ position of the compound formed in the steps such that precursor 1 is made; or alternatively using compound 6 as a starting material and: protecting 3′ and 5′ OH groups of compound 6 using a ethoxyvinylether; selectively deprotecting a tert-butyldimethylsilyl group of a compound formed in the prior step; and introducing a triflate leaving group in the 2′ position of the compound formed in prior step, such that precursor 3 is made.

The present disclosure also provides packaged compositions including the precursor compounds and/or intermediates to the labeled compounds (e.g., precursor 1 or precursor 3) and instructions for making the labeled compounds and methods of use (e.g., written instructions for their use). The kit can further include appropriate buffers and reagents known in the art. Illustrative methods and materials that can be adapted for use with the invention disclosed herein are found in U.S. Pat. No. 9,011,817, the contents of which are incorporated herein by reference.

Embodiments of this disclosure also include methods of imaging cells or organs using a PET probe made according to the methods disclosed herein. For example, embodiments of the PET ¹⁸F labeled compounds can be used to image their localization and/or quantity of in subjects (e.g., in a human organ such as the heart, human cells such as a T Cells and/or T lymphoblasts, or a human organelle such as a mitochondria). The labeled compounds can be administered to the subject and then the subject or a portion of the subject can be imaged using a device such as Positron Emission Tomography (PET) to detect the presence and location within the subject, and/or quantity of the labeled compounds present. The presence and/or quantity can be used to detect the presence, location, and/or number/size of T Cells and/or T lymphoblasts at one or more locations in the subject. Administration of the compounds (e.g., compositions, pharmaceutical compositions, and the like) can be via any of the accepted modes of administration for diagnostic agents. These methods include oral, parenteral, rectal, vaginal, nasal, inhaled, topical (including transdermal), parenterally, subcutaneous and other systemic modes. Accordingly, an embodiment of the present disclosure is directed to a pharmaceutical composition comprising a pharmaceutically acceptable carrier or excipient and a diagnostically effective amount of a compound of the present disclosure. Optionally, such pharmaceutical compositions may contain other diagnostic and/or therapeutic and/or formulating agents if desired.

As noted above, embodiments of the invention include compounds where one or more atoms on the compound is coupled to a protecting group (“PG”) and/or coupled to a leaving group (“LG”). As is known in the art, a leaving group is typically a molecular moiety that departs with a pair of electrons in heterolytic bond cleavage. Leaving groups can be anions, cations or neutral molecules that are able to stabilize the additional electron density that results from bond heterolysis. As is known in the art, a protecting group is a molecular moiety that is typically introduced into a molecule by chemical modification of a functional group in order to obtain chemoselectivity in a subsequent chemical reaction. In illustrative embodiments of the invention, an LG on a compound of the invention comprises a triflate leaving group, and a PG on a compound of the invention comprises a tert-Butyloxycarbonyl alcohol protecting group; a tert-Butyloxycarbonyl amine protecting group; a tetrahydropyranyl alcohol protecting group; and/or ethoxyethyl alcohol protecting group.

In certain embodiments of the invention, Tr is trityl protecting group (PG), and Tf is triflate leaving group (LG). Alternative protecting groups (PG) that can be used include protecting groups such as benzyl (Bn, Bnl), 6-methoxyethoxymethyl ether (MEM), methoxymethyl ether (MOM), p-methoxybenzyl ether (PMB), methylthiomethyl ether, methoxytrityl (MMT), pivaloyl (piv), tetrahydropyranyl (THP), trimethylsilyl (TMS), acetyl, difluoroacetyl, trifluoroacetyl, isobutyryl, benzoyl, 9-fluorenylmethoxycarbonyl, phenoxyacetyl, dimethylformamidine, N,N-diphenyl carbamate, acetals prepared from acyclic or cyclic alkyl vinyl or substituted alkyl vinyl ethers (including but not limited to ethyl vinyl ether, ethyl isopropenenyl ether, dihydropyran, 2-alkyldihydropyran, dihydrofuran, 2-alkyldihydrofuran, 1-methoxycyclohexene, 5,6-Dihydro-4-methoxy-2H-pyran) or the like. Alternative leaving groups (LG) that can be used include tosylate, mesylates, alkylmsesylates, phenylsulfonates, nosylate, brosylate, acetate, alkyl acetates, phenylacetates, iodide, bromide, chloride, or the like. Alternative groups that can be substituted for the acetyl group include, carbobenzyloxy (cbz), p-methoxybenzyl carbonyl (Moz), tet-Butyloxycarbonyl (BOC), 9-fluorenylmethoxycarbonyl, phenoxyacetyl, dimethylformamidine, N,N-diphenyl carbamate, or the like. Alternative groups that can be substituted for the benzoyl group include, acetyl, difluoroacetyl, trifluoroacetyl, isobutyryl, 9-fluorenylmethoxycarbonyl, phenoxyacetyl, dimethylformamidine, N,N-diphenyl carbamate, or the like. Thus, for each of the synthesis described, each of the PG, LG, Ac, and/or Bz can be substituted as described above and herein.

The isotope used in embodiments of the invention can be an isotope such as ¹⁸F, ¹³¹I, ¹²⁵I, ¹²⁴I, ¹²³I, ¹²¹I, ⁷⁷Br, ⁷⁵Br, or ⁷⁵Br. The compound containing an isotope can include [¹⁸F]KF, [¹³¹I]NaI, [¹²⁵I]NaI, [¹²⁴I]NaI, [¹²³]NaI, [¹²¹I]NaI, [⁷⁷Br]NaBr, [⁷⁷Br]Br₂, [⁷⁵Br]NaBr, or [⁷⁵Br]Br₂. The amount of the precursor or starting material can be about 2 to 15 mg, which can be adjusted, along with other similar variables, depending on the amount of end product desired and the desired scale up of the synthesis. The reaction can include appropriate solvents, reactive compounds, buffers, and the like. For example, typically the isotope is ¹⁸F or another isotope disclosed in disclosed in U.S. Pat. No. 9,011,817. The fluorination reaction can be conducted at a temperature of about 70 to 165° C. In regard to the amounts of the compounds used, the amount used can be scaled up or scaled down depending on the amount of the chemical desired to be produced.

A variety of solvents suitable for the compounds of the invention are known in the art. In addition to acetonitrile, solvents such as DMSO, dimethylformamide, tertamyl alcohol and combinations thereof can be used. Also, solvents such as THE, dioxane, Sulfone and combinations thereof can be used.

In certain embodiments, compounds of the invention can be modified via chemical reaction. After cooling the reaction mixture at room temperature, it can be subjected to acid deprotection with some type of acid, for example with about 1N HCl (or some other acid) at about 85 C. for about 10 min. Finally, after cooling the resulting reaction mixture at room temperature, the mixture can be neutralized to s pH of about 6-7 and the resulting solution was injected into a C18 reveres phase HPLC column to separate out the final products. Also, an alternative to C18 reverse phase HPLC column purification is the use of series of C18 cartridges.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

1. A method of making a PET probe composition, the method comprising:
reacting a compound including an isotope with precursor 1: 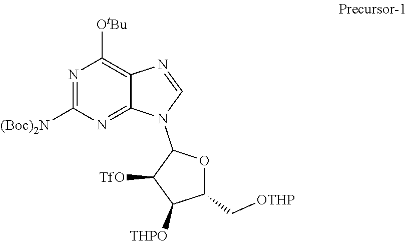
wherein:
^{t}Bu comprises a tert-Butyloxycarbonyl alcohol protecting group;
Boc comprises a tert-Butyloxycarbonyl amine protecting group;
THP comprises a tetrahydropyranyl alcohol protecting group;
EOE comprises an ethoxyethyl alcohol protecting group; and
Tf comprises a triflate leaving group:
wherein said reacting removes protecting group and leaving group moieties and couples the isotope to the compound;
so that the PET probe composition is made, wherein the PET probe composition comprises:

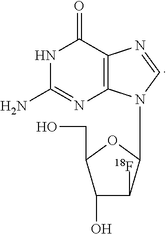

2. The method of claim 1, wherein the isotope is ¹⁸F.

3. The method of claim 1, wherein the compound including the isotope is [¹⁸F]KF.

4. The method of claim 1, wherein reacting occurs in a solvent selected from the group consisting of: dimethyl sulfoxide (DMSO), acetonitrile, dimethylformamide, and a combination thereof.

5. The method of claim 1, wherein the method comprises using: 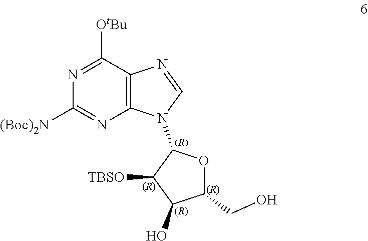
(compound 6) as a starting material, and:
(a) protecting 3′ and 5′ OH groups of compound 6 using a tetrahydropyranyl ether;
(b) selectively deprotecting a tert-butyldimethylsilyl group of a compound formed in step (a); and introducing a triflate leaving group in the 2′ position of the compound formed in step (b) such that precursor 1 is made.

6. A PET probe composition made by the method of claim 1.

7. A method of making a PET probe composition, the method comprising:
reacting a compound including an isotope with precursor 3: 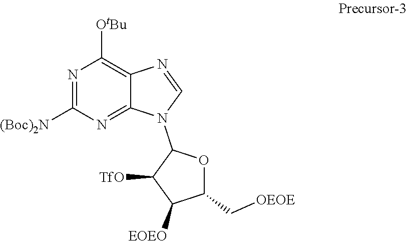
wherein:
^{t}Bu comprises a tert-Butyloxycarbonyl alcohol protecting group;
Boc comprises a tert-Butyloxycarbonyl amine protecting group;
THP comprises a tetrahydropyranyl alcohol protecting group;
EOE comprises an ethoxyethyl alcohol protecting group; and
Tf comprises a triflate leaving group:
wherein said reacting removes protecting group and leaving group moieties and couples the isotope to the compound;
so that the PET probe composition is made, wherein the PET probe composition comprises:

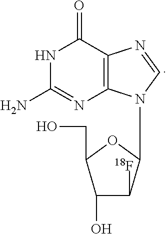

8. The method of claim 7, wherein the isotope is ¹⁸F.

9. The method of claim 7, wherein the compound including the isotope is [¹⁸F]KF.

10. The method of claim 7, wherein reacting occurs in a solvent selected from the group consisting of: dimethyl sulfoxide (DMSO), acetonitrile, dimethylformamide, and a combination thereof.

11. The method of claim 7, wherein the method comprises using: 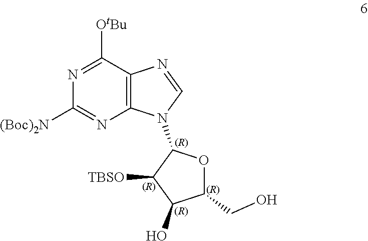
(compound 6) as a starting material, and:
(a) protecting 3′ and 5′ OH groups of compound 6 using an ethoxyvinylether ether,
(b) selectively deprotecting a tert-butyldimethylsilyl group of a compound formed in (a); and introducing a triflate leaving group in the 2′ position of the compound formed in step (b) such that precursor 3 is made.

12. A PET probe composition made by the method of claim 7.
